(19) United States
(12) Patent Application Publication
(10) Pub. No.: US 2006/0035286 A1
(43) Pub. Date: Feb. 16, 2006

(54) Monitor protein for measuring processing of protein

(76) Inventors: Yoshihiro Ohmiya, Osaka (JP); Emiko Ashitaka, Osaka (JP); Seji Ito, Osaka (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/537,971

(22) PCT Filed: Dec. 11, 2003
(86) PCT No.: PCT/JP03/15828

§ 371 Date: Jun. 9, 2005

(30) Foreign Application Priority Data
Dec. 12, 2002 (JP) 2002360744

(51) Int. Cl. G01N 33/567; C07H 21/04; C12P 21/06; C12N 5/08; C07K 14/435

(52) U.S. Cl. 435/7.2; 435/69.1; 435/320.1; 435/366; 530/350; 536/23.5

(57) ABSTRACT

A monitor protein for measuring processing of a protein comprising a processing region which contains an amino acid residue or an amino acid residue sequence to be processed and a secretory chimeric protein which originates in the stereostructure of a protein containing at least a processing region formed by the processing and shows a change in luminescent and fluorescent properties.

### TECHNICAL FIELD

The present invention relates to a monitor protein comprising a region at which the protein is cleaved by processing and a property variable region in which an energy transfer property is changed by the cleavage, a gene encoding the same, a processing cell and protein using the produced monitor protein or a method for screening a compound which facilitates or inhibits the processing.

### BACKGROUND ART

A protein which is a transcription product of a gene has sometimes a function directly, but in many cases, a protein which is a posttranslational product is activated through various modification processes such as sugar addition phosphorylation and processing. Among them, in a processing process, an active peptide is cut off from a preproprotein by being cleaved at a given site.

The processing process in which the active peptide is cut off from the preproprotein has been conventionally detected by an antibody specific for the active peptide after the cleavage or a direct change of molecular weights by electrophoresis or mass spectrometry. However, these methods are not simple, and are not procedures by which the processing is directly observed.

Light-emitting enzymes are effective as procedures to directly observe an intracellular transcription activity of a gene, and have been utilized as monitor proteins for detecting the gene expression. A fluorescent protein has a fluorescent activity without need of a cofactor nearly simultaneously with its intracellular expression. The fluorescent protein has been utilized as a monitor protein for intracellular localization of a protein using its fluorescent activity as an indicator.

A technique to track the changes of intermolecular or intramolecular interaction has been developed using energy transfer between a fluorescent protein and a fluorescent protein or a light-emitting enzyme and a fluorescent protein which are distinct proteins as an indicator. Monitor proteins have been constructed for analyzing the change of intracellular calcium using the energy transfer between the fluorescent protein and the fluorescent protein as the indicator and for analyzing phosphorylation of the protein using the energy transfer between the light-emitting enzyme and the fluorescent protein as the indicator. However, there is no available monitor protein which visualizes and quantifies the processing process using the energy transfer as the indicator.

It is an object of the present invention to develop a monitor protein capable of visualizing the processing process, a method for optimization thereof, a method for screening processing enzymes using the monitor protein and a method for screening medicaments which inhibit or facilitate a processing enzyme activity as well as utilize them for the treatment of diseases, examinations and new drug development.

### DISCLOSURE OF THE INVENTION

If a monitor protein construct where a processing region is inserted between a light-emitting enzyme and a fluorescent protein which can transfer energy is made, energy transfer is changed before and after the processing and a luminescence spectrum is also changed therewith. By the use of this spectral change as an indicator, it is possible to quantify an active peptide after the processing, quantify a processing enzyme activity and analyze the localization of a processing site, further screen a gene and a protein which control the processing enzyme activity as well as it becomes possible to utilize them for the treatment of various diseases and new drug development (development of medicaments which facilitate or inhibit the processing) using the processing as the indicator.

As a result of an extensive study for solving the above problems, the present inventor has made a monitor protein which has a processing cleavage region cleaved by the processing and a property variable region which exhibits a change in luminescent and fluorescent energy property by the processing.

The present invention relates to the following monitor proteins, genes encoding the same, processing cells and proteins using the produced monitor proteins, or methods for screening compounds which facilitate or inhibit the processing.

[1] A monitor protein capable of measuring processing of a protein, comprising one or more processing cleavage regions containing an amino acid residue or an amino acid sequence to be processed and one or more property variable regions which exhibit a change in luminescent and fluorescent energy property by the processing.

[2] The monitor protein according to [1] wherein the property variable region comprises a luminescent protein and a fluorescent protein.

[3] The monitor protein according to [1] wherein the monitor protein is a secretory protein.

[4] The monitor protein according to [1] wherein a processing enzyme which cleaves the processing cleavage region is any one selected from the group consisting of PC1, PC2, furin, proteasome, cathepsin and thrombin.

[5] The monitor protein according to [4] wherein the processing enzyme is PC1.

[6] The monitor protein according to [1] wherein the processing cleavage region is located between the luminescent protein and the fluorescent protein which constitute the property variable region.

[7] The monitor protein according to [1] wherein the luminescent protein is luciferase derived from any one selected from the group consisting of Cypridina noctiluca, Acanthephya purpurea, luminescent insects (firefly, headlight beetle, etc.), luminescent Pyrocystis lunula, luminescent earthworm, Latia neritoides, Renilla, and Aequorea Victoria (aequorin).

[8] The monitor protein according to [1] wherein the fluorescent protein is derived from any one selected from the group consisting of a green fluorescent protein (GFP), a yellow fluorescent protein (YFP), a blue fluorescent protein (BFP), a cyan fluorescent protein (CFP), DsRED and a red fluorescent protein (RFP).

[9] The monitor protein according to [2] characterized in that the luminescent protein in the property variable region is a light-emitting enzyme from secretory Cypridina noctiluca and the fluorescent protein in the property variable region is a yellow fluorescent protein from a mutant derived from luminescent Aequorea victoria.

[10] The monitor protein according to [1] wherein the processing cleavage region is composed of a sequence of 6 to 100 amino acids including a cleavage point cleaved by the processing enzyme.

[11] The monitor protein according to [1] having any of the following amino acid sequences (a) and (b):

(a) the monitor protein represented by an amino acid sequence in SEQ ID NO:2; and

(b) the monitor protein having one or more amino acid substitutions, additions, deletions or insertions in the amino acid sequence in SEQ ID NO:2, wherein the energy transfer property between the luminescent protein and the fluorescent protein and the cleavage activity by the processing enzyme in the processing cleavage region are retained.

[12] The monitor protein according to [1] wherein the processing cleavage region is SEQKQLQKRFGGFTGG.

[13] DNA encoding the monitor protein according to any of [1] to [12].

[14] The DNA according to [13], having any of the following DNA sequences (c) and (d):

(c) the DNA represented by a base sequence in SEQ ID NO:1; and

(d) the DNA which hybridizes with the DNA represented by the base sequence in SEQ ID NO:1 or DNA complementary thereto under a stringent condition, wherein the protein encoded by the DNA retains the energy transfer property between the luminescent protein and the fluorescent protein and the cleavage activity by the processing enzyme in the processing cleavage region.

[15] An expression vector comprising the DNA according to [13].

[16] A method for measuring a processing ability of a certain cell characterized by introducing any of the monitor protein according to any of [1] to [12], the DNA according to [13] to [14] and the expression vector according to [15] into the cell, and quantitatively evaluating a change in energy transfer property of the monitor protein.

[17] The method according to [16] wherein said cell is a cell derived from human.

[18] A method for measuring a processing ability of a subject protein comprising a step of reacting the subject protein with the monitor protein according to any of [1] to [12] and a step of measuring a change in energy transfer property of the monitor protein.

[19] A method for screening a processing enzyme, comprising a step of reacting a subject protein with the monitor protein according to any of [1] to [12] and a step of selecting the subject protein which changes a property of the monitor protein by measuring a change in energy transfer property of the monitor protein before and after the reaction with the subject protein.

[20] A method for screening a compound which facilitates or inhibits processing, comprising a step of contacting a subject sample and a processing enzyme and the monitor protein according to any of [1] to [12] and a step of selecting a sample which facilitates or inhibits the property by measuring a change in energy transfer property of the monitor protein before and after the contact with the subject sample.

[21] A method for screening a compound which facilitates or inhibits processing (enzyme activity), comprising a step of preparing a cell into which any of the monitor protein according to any of [1] to [12], the DNA according to [13] to [14] and the expression vector according to [15] is introduced, and a step of selecting a sample which facilitates or inhibits a property change by measuring the property change in energy transfer of the monitor protein expressed in the cell in the presence or absence of the subject sample.

The present specification further discloses the following invention.

1. Proteins showing in the following (1) to (4):

(1) a monitor protein represented by an amino acid sequence in SEQ ID NO:2;

(2) a monitor protein wherein a sequence or a position in a region cleaved by processing and a property variable region (a luminescent protein and a fluorescent protein) is changed in the amino acid sequence in SEQ ID NO:2;

(3) a monitor protein wherein a processing cleavage region figured by an underline in the amino acid sequence in SEQ ID NO:2 (amino acid sequence of 18 amino acid residues including a cleavage point of prepronocistatin protein) is substituted with 10 to 100 amino acid residues, preferably 20 to 40 amino acid residues in the vicinity of a processing cleavage site; and

(4) a monitor protein wherein a property is variable in a property variable region due to a change of a three dimensional structure in the vicinity of the processing cleavage region. The monitor protein wherein a luminescent protein and a fluorescent protein are arranged with sandwiching the processing region, the energy transfer from the luminescent protein to the fluorescent protein is not effected before and after the processing, and thus a luminescent color is changed is suitable.

In a preferred embodiment, in the monitor protein, the luminescent protein and the fluorescent protein are arranged at both termini of the property variable region, respectively. In the monitor protein, either one may be present at the N-terminus, but it is more preferable that the luminescent protein and the fluorescent protein are present in this order from the N-terminus. In the monitor protein having the luminescent protein and the fluorescent protein, when the processing does not occur, the monitor protein emits yellow-green light by the energy transfer of blue light emitted from the luminescent protein (secretory Cypridina noctiluca, light-emitting enzyme) to the fluorescent protein mutant (EYFP). When the processing has occurred, the energy transfer is not effected and only the blue light is emitted from the monitor protein. The processing can be analyzed using this color change of the emitted light in the property variable region as the indicator.

Conventionally, there have been the monitor proteins by which intracellular phosphorylation and calcium dynamics can be observed. However, the present inventor has demonstrated the monitor protein capable of visualizing and quantifying the processing process for the first time. A system for analyzing cellular functions has been designed using the change in the energy transfer between the fluorescent protein and the fluorescent protein or between the luminescent protein and the fluorescent protein as the indicator. However, the present researcher has utilized it for a system which analyzes the processing for the first time.

The present invention will be described in more detail below.

The monitor protein of the present invention is characterized by comprising the processing cleavage region and the property variable region.

The processing cleavage region is a region comprising two or more amino acids, which is cleaved by the processing enzyme, and the amino acid sequence thereof is changed depending on a type of the processing enzyme subjected to be monitored.

In the present invention, the processing cleavage region is the region comprising the amino acid sequence capable of being recognized and cleaved by the processing enzyme, comprises one or more amino acids, and the amino acid sequence thereof is changed depending on the type of the processing enzyme subjected to be monitored. For example, in the monitor protein represented by SEQ ID NO:2, PC1 which is the processing enzyme recognizes and cleaves a cleavage point, “KR”, but the cleavage occurs at only the processing cleavage region although there are four “KR” in SEQ ID NO:2 (two in a luciferase moiety, one in the processing cleavage region, and one in YFP). Therefore, the processing cleavage region may comprise not only the cleavage point (KR) but also further an additional sequence having an auxiliary portion for the enzyme to recognize and be able to cleave the cleavage point, which scarcely or does not affect the cleavage at all. One example of the processing cleavage region includes “SEQKQLQKRFGGFTGG” for the processing enzyme (PC1).

The processing cleavage region will be illustrated with reference to PC1 as the processing enzyme.

The cleavage recognition sequence of PC1 is Lys-Arg, and the processing cleavage region contains Lys-Arg and further links 3 to 50, preferably 4 to 40 and more preferably 5 to 20 amino acids at the C-terminus and the N-terminus. The amino acids used for the processing cleavage region are preferably usual amino acids such as Gly, Ala, His, Arg, Lys, Ser, Cys (including cystine), Thr, Met, Phe, Tyr, Trp, Leu, Ile, Val, Glu, Asp, Gln, Asp, Pro which constitute proteins, but may be other natural or synthetic amino acids such as Sar, β-alanine, norvaline, ornithine and cysteic acid.

The above is the case where the processing enzyme is PC1, and the processing cleavage region for the other processing enzyme can be also designed in a similar manner.

The total number of the amino acids in the cleavage region is about 6 to 100, preferably about 8 to 50, and more preferably about 10 to 40.

The processing enzyme is not particularly limited as long as it exclusively degrades a protein precursor synthesized in vivo and converts into a mature protein, and for example, PC1, PC2, furin, proteasome, cathepsin and thrombin are exemplified.

The processing enzymes and the cleavage regions thereof in addition to PC1 are exemplified below.

TABLE 1

| | Amino acid |
|---|---|
| | sequence at |
| | cleavage point |
| Processing enzyme | of processing |
| PC1 | KR↓ |
| PC2 | KR↓ |
| Furin | RRKR↓ |
| Proteasome | KM↓ |
| Cathepsin | KM↓ |
| Thrombin | LVPR↓ |

For example, PC1, PC2 and furin cleave a C-terminal protein of KR, and a particular processing enzyme becomes possible to cleave by changing a structure of the sequence in the vicinity of KR or a structure of a whole protein.

When the processing enzyme subjected to be monitored is one, one cleavage recognition sequence may be included. When multiple processing enzymes are simultaneously monitored, it is possible to introduce different cleavage recognition sequences corresponding to two or more processing enzymes into the cleavage region. A compounds which regulates only a particular processing enzyme activity can be screened by constructing the cleavage region having only the cleavage recognition sequence of the particular processing enzyme and having no cleavage recognition sequence of the other processing enzyme. Even when one processing enzyme which acts upon multiple substrates is subjected, a compound which facilitates or inhibits the processing of the particular substrate can be screened by selecting the cleavage recognition sequence or the sequence in the vicinity thereof in accordance with the sequence of the particular substrate.

In the property variable region, the energy transfer property may be changed when cleaved by the processing enzyme, and for example, the combination of the luminescent protein with the fluorescent protein and the combination of the luminescent protein with a colored protein can be included.

When the luminescent protein and the fluorescent protein are linked through the processing cleavage region, before the processing cleavage region is cleaved, the energy transfer from the luminescent protein to the fluorescent protein occurs, but after being cleaved, this energy transfer does not occur. Thus, it is advantageous in that the presence or absence of the cleavage can be easily quantified by measuring the energy transfer. It is preferable that the luminescent protein and the processing cleavage region and the fluorescent protein are linked in the order of the luminescent protein—the processing cleavage region—the fluorescent protein, but may be linked in the order of the fluorescent protein—the processing cleavage region—the luminescent protein as long as a luminescent/fluorescent property is changed by the cleavage by the processing enzyme.

The processing cleavage region may also be introduced into a site which does not crucially affect the luminescent property in the luminescent protein. In this case, the luminescent protein may lose the luminescent property after the processing.

Likewise, the processing cleavage region may also be introduced into a site which does not crucially affect the fluorescent property in the fluorescent protein. In this case, the fluorescent protein may lose the fluorescent property after the processing.

As the luminescent protein, luciferases derived from various luminescent organisms such as Cypridina noctiluca, Acanthephya purpurea, luminescent insects (firefly, headlight beetle, etc.), luminescent earthworm, Latia neritoides, Renilla, and Aequorea victoria (aequorin) are exemplified. For example, the Cypridina noctiluca luciferase is a secretory type, and is preferable because the monitor protein also becomes secretory when this is used. When the secretory monitor protein is used, a level of the processing which occurs in living cells can be evaluated without disrupting the cells. In the case of non-secretory luciferase such as luciferase derived from Renilla, it can be utilized as a secretory luminescent protein by introducing a secretory protein into the N-terminal side.

As the fluorescent protein, a green fluorescent protein (GFP), a yellow fluorescent protein (YFP), a blue fluorescent protein (BFP), a cyan fluorescent protein (CFP), DsRED and a red fluorescent protein (RFP) are exemplified. As the colored protein, a colored blue protein (phycocyanin) and a red colored protein (phycoerythrin) are exemplified. Phycocyanin and phycoerythrin can absorb the light of firefly luciferase.

The fluorescent protein of the present invention is selected so that the light released from the biological luminescent protein is an excitation wavelength of the fluorescent protein. Such a combination includes, for example, the followings.

TABLE 2

| Biological luminescent | Fluorescent protein |
|---|---|
| protein | or colored protein |
| Cypridina noctiluca | GFP, YFP, BFP, CFP, |
| luciferase | DsRED, RFR |
| Firefly luciferase | DsRED, Phycocyanin, |
| | Phycoerythrin |
| Luminescent Pyrocystis | GFP, YFP, BFP, CFP, |
| lunula luciferase | DsRED, RFP |
| Headlight beetle | DsRED |
| luciferase | |
| Renilla luciferase | GFP, YFP, BFP, CFR |
| | DsRED, RFP |
| Aequorin | GFP, YFP, BFP, CFP, |
| | DsRED, RFP. |

In one preferable embodiment, as the monitor protein of the invention, the proteins shown in the following (1) to (3) are exemplified.

(1) The monitor protein represented by the amino acid sequence in SEQ ID NO:2.

(2) The monitor protein represented by the amino acid sequence having one or more amino acid deletions, substitutions or additions in the amino acid sequence in SEQ ID NO:2, and which has the light-emitting enzyme activity, the fluorescent protein activity and the energy transfer activity.

(3) The monitor protein encoded by the DNA represented by the base sequence in SEQ ID NO:1 or the DNA which hybridizes with. the DNA complementary thereto under the stringent condition, and which has the light-emitting enzyme activity, the fluorescent protein activity and the energy transfer activity.

The protein (2) is the protein where mutations to an extent that the light-emitting enzyme activity, the fluorescent protein activity and the energy transfer activity are not lost are introduced into the protein (1). Such mutations naturally occur and further encompass artificial mutations. Procedures by which induce the artificial mutation include, but are not limited to site-directed mutagenesis (Nucleic Acids Res., 10:6487-6500, 1982). The number of mutated amino acids are not limited as long as the secretion, the luminescence, the fluorescent activity and the energy transfer property are not lost, and is typically within 20 amino acids, preferably within 15 amino acids, more preferably within 10 amino acids and most preferably within 5 amino acids. Whether the protein into which the mutation is introduced keeps the luminescent/fluorescent activity can be determined by examining the luminescent/fluorescent activity of the protein.

The protein (3) is the monitor protein obtained by utilizing the hybridization of DNA, and which has the secretory type light-emitting enzyme activity, the fluorescent protein activity and the energy transfer activity. The “stringent condition” in the protein (3) refers to the condition where only the specific hybridization occurs whereas no non-specific hybridization occurs. Such a condition is typically the condition of “1×SSC and 0.1% SDS at 37° C.”, preferably “0.5×SSC and 0.1% SDS at 42° C.”, and more preferably “0.2×SSC and 0.1% SDS at 65° C”. The DNA obtained by the hybridization typically has high homology to the DNA represented by the base sequence in SEQ ID NO:1. The high homology indicates 60% or more homology, preferably 75% or more homology and more preferably 90% or more homology.

The protein of the present invention can be obtained by incorporating a gene of the present invention described later into an expression vector and expressing it in appropriate host cells. As the expression vector, for example, pBT-VL-b-YFP and the like can be used. As the host cell, Escherichia coli BL21 strain and the like can be used.

The gene of the present invention includes the genes represented by the following (1) and (2).

(1) The gene represented by the base sequence in SEQ ID NO:1.

(2) The DNA sequence which hybridizes with the DNA represented by the base sequence in SEQ ID NO:1 or the DNA complementary thereto under the stringent condition.

The protein encoded by the above DNA is the protein obtained by utilizing the hybridization of DNA one another, and which has the secretory type light-emitting enzyme activity, the fluorescent protein activity and the energy transfer activity.

The “stringent condition” herein refers to the condition where only the specific hybridization occurs whereas no non-specific hybridization occurs. Such a condition is typically the condition of “1×SSC and 0.1% SDS at 37° C.”, preferably “0.5×SSC and 0.1% SDS at 42° C.”, and more preferably “0.2×SSC and 0.1% SDS at 65° C.”. The DNA obtained by the hybridization typically has high homology to the DNA represented by the base sequence in SEQ ID NO:1 or 2. The high homology indicates 60% or more homology, preferably 75% or more homology, more preferably 90% or more homology, and particularly 95% or more homology.

The protein of the present invention can be obtained by incorporating the gene of the present invention described later into the expression vector and expressing it in appropriate host cells. As the expression vector, for example, pBT-VL-mp-YFP (VL, mp and YFP indicate Cypridina noctiluca luciferase, a monitor peptide and the yellow fluorescent protein, respectively) and the like can be used. The host cells include eukaryotic cells such as mammalian cells and yeast cells, and prokaryotic cells such as Escherichia coli, Bacillus subtilis, algal and fungal cells, and any of them may be used. As the preferable host cell, mammalian cultured cell COS7 cell line (in this system, it is important to go through protein synthesis and protein modification processes of a mammalian system, i.e., this process will be monitored) and the like can be used.

A gene (polynucleotide) encoding a preferable chimera protein of the present invention is

(1) the gene having the base sequence in SEQ ID NO:1 or

(2) the gene having the DNA represented by the base sequence in SEQ ID NO:1 or the DNA which hybridizes with the DNA complementary thereto under the stringent condition.

(Screening Method I)

The change in the energy transfer property of the monitor protein is quantitatively evaluated by introducing any of the monitor protein, the DNA sequence or the expression vector into certain cells.

The method enables to quantitatively evaluate the processing ability of the cell.

(Screening Method II)

The processing ability of a subject protein is quantitatively evaluated by reacting the subject protein with the monitor protein of the present invention and measuring the change in the energy transfer property of the monitor protein.

In the method, what processing enzyme the subject protein is or what processing enzyme activity is strong can be quantitatively measured by typically preparing multiple monitor proteins and analyzing what monitor protein changes the energy transfer property by the subject protein.

(Screening Method III)

A new processing enzyme can be screened by performing a step of reacting a subject protein with the monitor protein of the present invention and a step of selecting the subject protein which changes the property of the monitor protein by measuring the change in the energy transfer property of the monitor protein before and after the reaction with the subject protein.

(Screening Method IV)

The method for screening a compound which facilitates or inhibits the processing in a cell free system, comprising a step of contacting a subject sample, the processing enzyme and the monitor protein of the present invention, and a step of selecting the sample which facilitates or inhibits the property by measuring the change in the energy transfer property of the monitor protein before and after the contact with the subject sample.

By the method, the compound which facilitates or inhibits the processing can be screened without considering factors such as migration into the cells.

The method for screening a compound which facilitates or inhibits the processing (enzyme activity), comprising a step of preparing cells into which any of the monitor protein, the DNA sequence encoding the monitor protein and the expression vector comprising the DNA sequence of the present invention has been introduced, and a step of selecting the sample which facilitates or inhibits the property by measuring the change in the energy transfer property of the monitor protein expressed in the cells in the presence or absence of the subject sample.

By the method, the compound which exerts medicinal efficacy by migrating into blood by oral administration can be screened.

The present invention provides the monitor protein capable of quantifying the novel processing, the gene encoding the same, and the gene which controls the expression of the present enzyme. By the use of the present monitor protein, the processing process which occurs in the cell can be visualized without disrupting the cell. These can be utilized for the treatment of diseases, the examination and the new drug development.

### BRIEF DESCRIPTION OF THE DRAWINGS

FIG. 1 shows constructs where a processing site (A), a mutated processing site (B) or deleted processing site (C) is inserted into an energy transferable secretory chimera protein.

FIG. 2 shows DNA sequences of regions where a processing site (A), a mutated processing site (B) or deleted processing site (C) is inserted into an energy transferable secretory chimera protein.

FIG. 3 shows Western blotting analysis of intracellular and extracellular samples of cells into which a secretory chimera protein where a processing sequence had been inserted was introduced, and detection results of changes in cleavage efficiency when a processing enzyme was introduced.

FIG. 4 shows that energy transfer efficiency and spectra are changed when a processing enzyme was introduced into cells which expressed a secretory chimera protein where a processing sequence had been inserted. (a) Vluc-NST/Nco-EYFP alone, (b) PC1 was expressed with Vluc-NST/Nco-EYFP, (c) PC2 was expressed with Vluc-NST/Nco-EYFP, and (d) Vluc.

FIG. 5 shows that energy transfer efficiency of a secretory chimera protein where a processing sequence had been inserted was largely reduced by a cleavage enzyme PC1, and also shows results of cleavage efficiency quantified by the results of the Western blotting.

Next, the present invention will be described in more detail with reference to the following Examples.

### BEST MODE FOR CARRYING OUT THE INVENTION

The present invention will be described in more detail using the following Examples.

### EXAMPLE 1

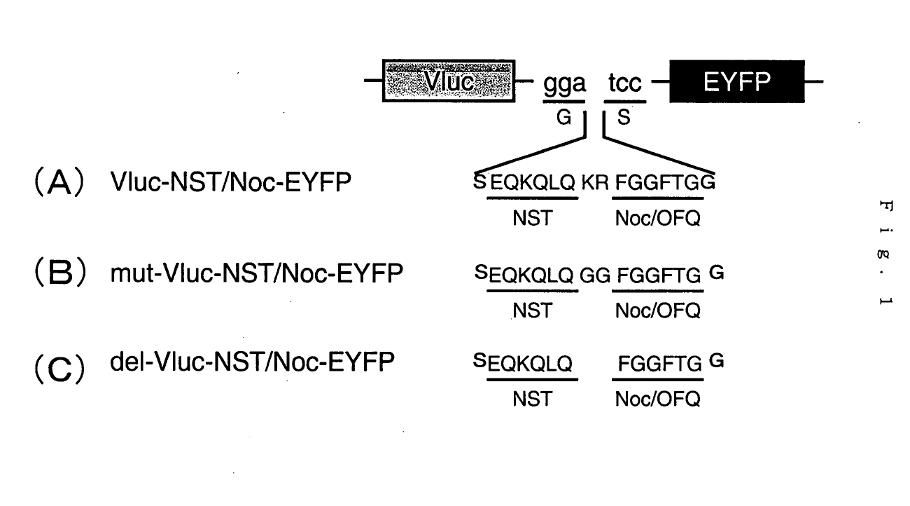

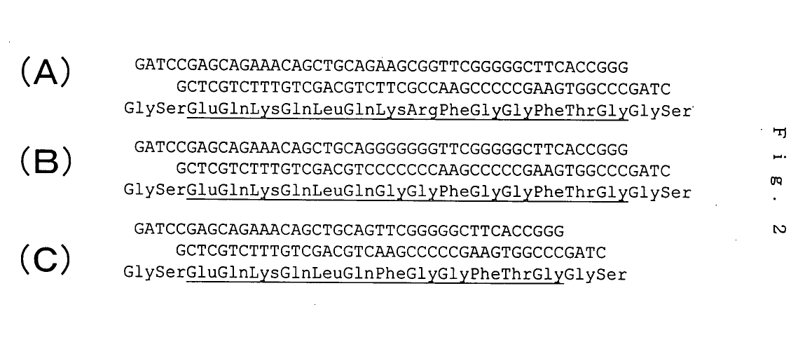

Nocistatin and nociceptin are hormone peptides cleaved off from the same preproprotein by a cleavage enzyme in the processing process, and are known to control reciprocal effects when pain runs in the body. A monitor protein where a nocistatin (described as NST in the figure) peptide sequence and a nociceptin (described as Noc in the figure) peptide sequence to be processed had been inserted in an insertion site of a secretory chimera protein where energy was transferable between a luminescent protein and a fluorescent protein was made (FIG. 1(A)). In conjunction therewith, a monitor protein where a peptide sequence Lys-Arg which was essential for cleavage recognition and was cleaved had been mutated to Gly-Gly (FIG. 1(B)), and a monitor protein where the peptide sequence was deleted (Fig, 1(C)) were made. They were referred to as Vluc-NST/Noc-EYFP, mut-Vluc-NST/Noc-EYFP and del-Vluc-NST/Noc-EYFP, respectively. A BamHI site which was an insertion restriction enzyme site of the monitor protein was cleaved, and subsequently chemically synthesized DNA sequences shown in FIG. 2 were ligated by a ligase reaction. The insertion was confirmed by DNA sequencing.

### EXAMPLE 2

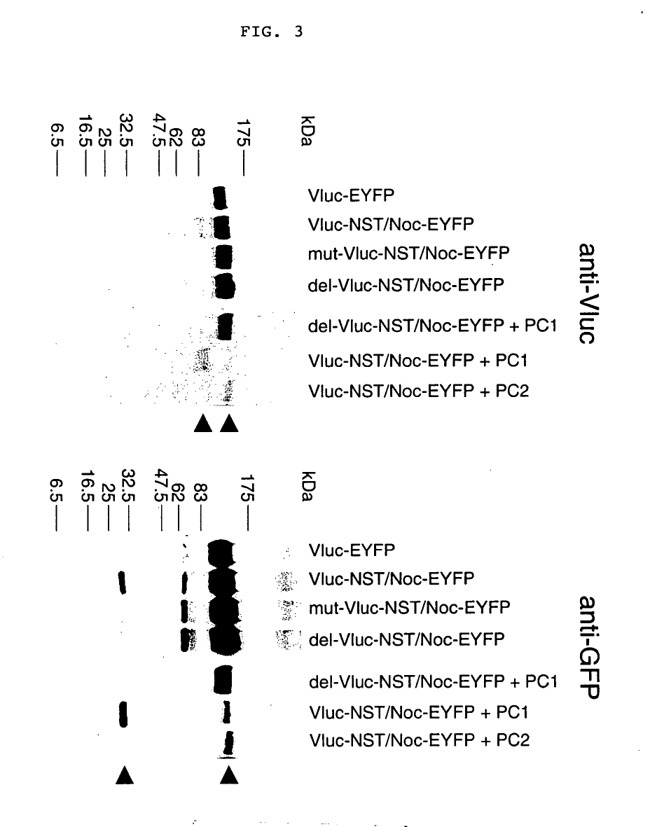

NG108-15 cells derived from nervous cells were transfected with gene vectors containing three monitor proteins, Vluc-NST/Noc-EYFP, mut-Vluc-NST/Noc-EYFP and del-Vluc-NST/Noc-EYFP using a commercially available transfection reagent. Proteins secreted in culture media were analyzed by a Western blotting method (FIG. 3). Vluc-EYFP which had no insertion sequence was used as a control. An anti-Cypridina noctiluca luciferase antibody (ant-Vluc) recognizes a light-emitting enzyme at the N-terminal side of the monitor protein, and four types of the monitor proteins have a molecular weight of 95 kDa. An anti-green fluorescent protein antibody (anti-GFP) recognizes the fluorescent protein at the C-terminal side of the monitor protein, and four monitor proteins showed the molecular weight of 95 kDa as a main protein. However, in the monitor protein of Vluc-NST/Noc-EYFP, a minor band at 27 kDa compared to the band at 95 kDa was observed. A processing enzyme was expressed in NG108-15 cells derived from the nervous cells, and cleaved the monitor protein, the antibody recognized EYFP at the C-terminal side after the cleavage and consequently this band was produced. In the mutated or deleted, two mut-Vluc-NST/Noc-EYFP and del-Vluc-NST/Noc-EYFP, this 27 kDa band was not detected and it has been demonstrated that the processing enzyme can strictly recognize the inserted amino acid sequence. But, a 68 kDa band recognized by anti-GFP seems to be artificial. In order to demonstrate that the cleavage which occurs in the monitor protein of Vluc-NST/Noc-EYFP is attributed to either PC1 or PC2 which is a representative processing enzyme which cleaves insulin and enkephalin, PC1 or PC2 was forcibly expressed in the cells into which the monitor protein had been transfected. As a result, it has been demonstrated that PC1 remarkably increased the 27 kDa band compared to the 95 kDa band whereas PC2 scarcely changed. Consequently, it has been demonstrated for the first time that the processing between nocistatin and nociceptin (NST/Noc) occurs due to PC1.

### EXAMPLE 3

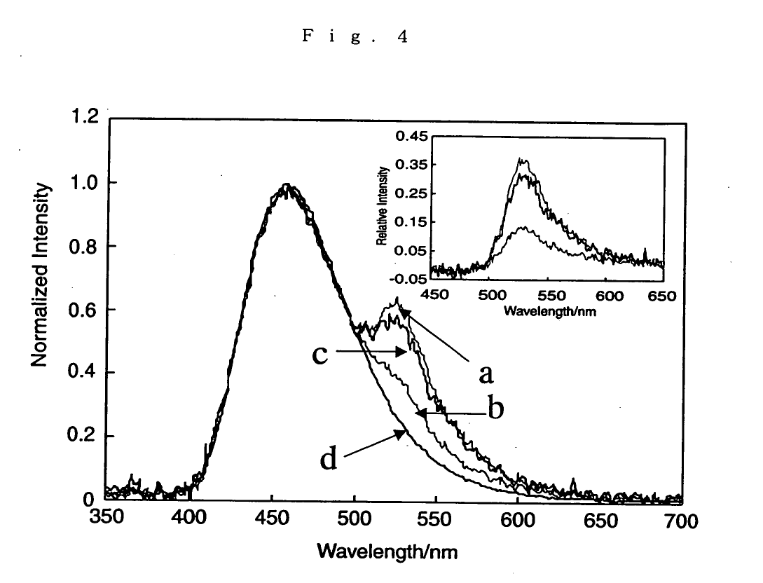

NG108-15 cells derived from nervous cells were transfected with gene vectors containing the monitor protein, Vluc-NST/Noc-EYFP and Vluc using a commercially available transfection reagent. In conjunction therewith, the processing enzyme, PC1 or PC2 was forcibly expressed in the cells which had expressed the monitor protein, Vluc-NST/Noc-EYFP. Luminescence spectra in culture media secreted by these four types of the cells were measured (FIG. 4). In the monitor protein, Vluc-NST/Noc-EYFP, a peak at 525 nm caused by the energy transfer which had occurred in the monitor protein could be observed in addition to a peak at 460 nm conformed to that of the luminescence spectrum from Vluc alone. In the culture medium of the cells where PC2 had been expressed in this monitor protein, Vluc-NST/Noc-EYFP, the spectrum was not changed whereas in the culture medium of the cells where PC1 had been expressed in Vluc-NST/Noc-EYFP, the peak at 525 nm was reduced. This reveals that the processing enzyme PC1 cleaved the monitor protein, Vluc-NST/Noc-EYFP and the energy transfer in the monitor protein did not occur as is also evident from the result of the Western blotting. That is, by measuring the luminescence spectrum in the culture medium, the processing level which occurs in the living cells can be evaluated without disrupting the cells.

### EXAMPLE 4

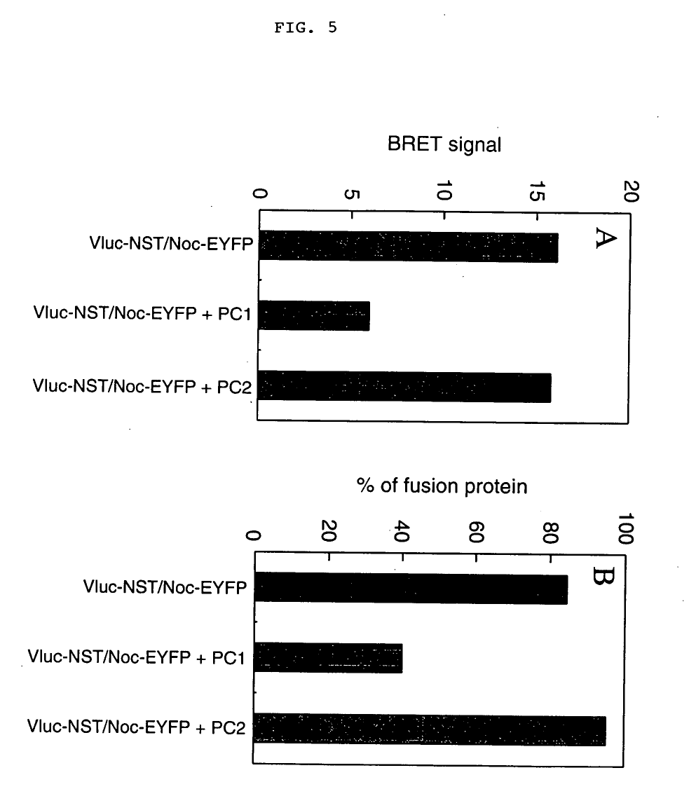

The cleavage efficiency of the monitor protein, Vluc-NST/Noc-EYFP transfected into NG108-15 cells derived from the nervous cells was detected by the Western blotting, and a mass of non-cleaved protein was quantified by an image analysis (FIG. 5B). Subtracted spectra were obtained by comparing the luminescence spectra of the monitor protein, Vluc-NST/Noc-EYFP with the spectrum of Vluc alone (inner figure in FIG. 4), and spectral areas were evaluated as energy transfer effects (BRET signal)(FIG. 5A). In the cases of the monitor protein, Vluc-NST/Noc-EYFP and the monitor protein, Vluc-NST/Noc-EYFP in which PC2 had been introduced, both the non-cleaved protein mass and the BRET signal shows that the cleavage did not occur. Meanwhile, in the case of the monitor protein, Vluc-NST/Noc-EYFP in which PC1 had been introduced, both the non-cleaved protein mass and the BRET signal shows that 60% monitor protein was cleaved. This reveals that the efficiency of the processing which occurs in the cells can be simultaneously evaluated by measuring the luminescence spectrum in the culture medium without quantifying by a method such as electrophoresis.

1. A monitor protein capable of measuring processing of a protein, comprising one or more processing cleavage regions containing an amino acid residue or an amino acid sequence to be processed and one or more property variable regions which exhibit a change in luminescent and fluorescent energy property by the processing.

2. The monitor protein according to claim 1 wherein the property variable region comprises a luminescent protein and a fluorescent protein.

3. The monitor protein according to claim 1 wherein the monitor protein is a secretory protein.

4. The monitor protein according to claim 1 wherein a processing enzyme which cleaves the processing cleavage region is any one selected from the group consisting of PC 1, PC2, furin, proteasome, cathepsin and thrombin.

5. The monitor protein according to claim 4 wherein the processing enzyme is PC1.

6. The monitor protein according to claim 1 wherein the processing cleavage region is located between the luminescent protein and the fluorescent protein which constitute the property variable region.

7. The monitor protein according to claim 1 wherein the luminescent protein is luciferase derived from any one selected from the group consisting of Cypridina noctiluca, Acanthephya purpurea, luminescent insects (firefly, headlight beetle, etc.), luminescent Pyrocystis lunula, luminescent earthworm, Latia neritoides, Renilla, and Aequorea victoria (aequorin).

8. The monitor protein according to claim 1 wherein the fluorescent protein is derived from any one selected from the group consisting of a green fluorescent protein (GFP), a yellow fluorescent protein (YFP), a blue fluorescent protein (BFP), a cyan fluorescent protein (CFP), DsRED and a red fluorescent protein (RFP).

9. The monitor protein according to claim 2 characterized in that the luminescent protein in the property variable region is a light-emitting enzyme from secretory Cypridina noctiluca and the fluorescent protein in the property variable region is a yellow fluorescent protein from a mutant derived from luminescent Aequorea victoria.

10. The monitor protein according to claim 1 wherein the processing cleavage region is composed of a sequence of 6 to 100 amino acids including a cleavage point cleaved by the processing enzyme.

11. The monitor protein according to claim 1 having any of the following amino acid sequences (a) and (b):
(a) the monitor protein represented by an amino acid sequence in SEQ ID NO:2; and
(b) the monitor protein having one or more amino acid substitutions, additions, deletions or insertions in the amino acid sequence in SEQ ID NO:2, wherein the energy transfer property between the luminescent protein and the fluorescent protein and the cleavage activity by the processing enzyme in the processing cleavage region are retained.

12. The monitor protein according to claim 1 wherein the processing cleavage region is SEQKQLQKRFGGFTGG.

13. DNA encoding the monitor protein according to claim 1.

14. The DNA according to claim 13, having any of the following DNA sequences (c) and (d):
(c) the DNA represented by a base sequence in SEQ ID NO: 1; and
(d) the DNA which hybridizes with the DNA represented by the base sequence in SEQ ID NO:1 or DNA complementary thereto under a stringent condition, wherein the protein encoded by the DNA retains the energy transfer property between the luminescent protein and the fluorescent protein and the cleavage activity by the processing enzyme in the processing cleavage region.

15. An expression vector comprising the DNA according to claim 13.

16. A method for measuring a processing ability of a certain cell characterized by introducing any of the monitor protein according to any of claims 1 to 12, the DNA according to claim 13 to 14 and the expression vector according to claim 15 into the cell, and quantitatively evaluating a change in energy transfer property of the monitor protein.

17. The method according to claim 16 wherein said cell is a cell derived from human.

18. A method for measuring a processing ability of a subject protein comprising a step of reacting the subject protein with the monitor protein according to claim 1 and a step of measuring a change in energy transfer property of the monitor protein.

19. A method for screening a processing enzyme, comprising a step of reacting a subject protein with the monitor protein according to claims 1 and a step of selecting the subject protein which changes a property of the monitor protein by measuring a change in energy transfer property of the monitor protein before and after the reaction with the subject protein.

20. A method for screening a compound which facilitates or inhibits processing, comprising a step of contacting a subject sample and a processing enzyme and the monitor protein according to claim 1 and a step of selecting a sample which facilitates or inhibits the property by measuring a change in energy transfer property of the monitor protein before and after the contact with the subject sample.

21. A method for screening a compound which facilitates or inhibits processing (enzyme activity), comprising a step of preparing a cell into which any of the monitor protein according to any of claims 1 to 12, the DNA according to claims 13 to 14, and the expression vector according to claim 15 is introduced, and a step of selecting a sample which facilitates or inhibits a property change by measuring the property change in energy transfer of the monitor protein expressed in the cell in the presence or absence of the subject sample.


SEQUENCE LISTING (5 sequences)

SEQ ID NO: 1 - DNA, 2502 bp, mammalian
atgaagataataattctgtctgttatattggcctactgtgtcaccgacaactgtcaagatgcatgtcctgtagaagcggaaccgccatcaagtacaccaacagttccaacttcttgtgaagctaaagaaggagaatgtatagataccagatgcgcaacatgtaaacgagatatactatcagatggactgtgtgaaaataaaccagggaagacatgctgtagaatgtgccagtatgtgattgaatgcagagtagaagcagctggttattttagaacgttttacggcaaaagatttaattttcaggaacctggtaaatatgtgctggctaggggaaccaagggtggcgattggtctgtaaccctcaccatggagaatctagatggacagaagggagctgtgctgactaagacaacactggaggttgcaggagacgtaatagacattactcaagctactgcagatcctatcacagttaacggaggagctgacccagttatcgctaacccgttcacaattggtgaggtgaccattgctgttgttgaaataccgggcttcaatatcacagtcatcgaattctttaaactaatcgtgattgatattctgggaggaagatctgtgagaattgctccagacacagcaaacaaaggactgatatctggtatctgtggtaatctggagatgaatgacgctgatgactttactacagatgcagatcagctggcgatccaacccaacataaacaaagagttcgacggctgcccattctatggcaatccttctgatatcgaatactgcaaaggtctgatggagccatacagagctgtatgtcgtaacaatatcaacttctactattacactctatcctgtgccttcgcttactgtatgggaggagaagaaagagctaaacacgtccttttcgactatgttgagacatgcgctgcgccggaaacgagaggaacgtgtgttttatcaggacatactttctatgacacattcgacaaagcaagatatcaattccagggcccatgcaaggagattctgatggccgcagactgttactggaacacatgggatgtaaaggtttcacatagagacgtcgaatcatacactgaggtagagaaagtaacaatcaggaaacagtcaactgtagtagatctcattgtggatggcaagcaggtcaaggttggaggagtggatgtatctatcccgtacagctctgagaacacttccatatactggcaggatggagacatcctgacgacggccatcctacctgaagctcttgtcgttaagttcaactttaagcagctccttgtagttcatatcagagatccattcgatggaaagacatgcggcatatgtggtaactataatcaagattcaactgatgatttctttgacgcagaaggagcatgcgctctaacccccaaccccccaggatgtacagaggaacagaaaccagaagctgagcgactttgcaataatctctttgattcttctatcgacgagaaatgtaatgtctgctacaagcctgaccggattgcccgatgtatgtacgagtattgcctgaggggacaacaaggattttgtgaccatgcttgggagttcaagaaagaatgctacataaaacatggagacactctagaagtaccacctgaatgtcaaggatccacagagcccggcctggaggaggtgggggagattgagcagaaacagctgcagaagcggttcgggggcttcaccggggcccggaagtcggcccggaagttggccaaccagggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 2 - protein, 833 aa, mammalian
MKIIILSVILAYCVTDNCQDACPVEAEPPSSTPTVPTSCEAKEGECIDTRCATCKRDILSDGLCENKPGKTCCRMCQYVIECRVEAAGYFRTFYGKRFNFQEPGKYVLARGTKGGDWSVTLTMENLDGQKGAVLTKTTLEVAGDVIDITQATADPITVNGGADPVIANPFTIGEVTIAVVEIPGFNITVIEFFKLIVIDILGGRSVRIAPDTANKGLISGICGNLEMNDADDFTTDADQLAIQPNINKEFDGCPFYGNPSDIEYCKGLMEPYRAVCRNNINFYYYTLSCAFAYCMGGEERAKHVLFDYVETCAAPETRGTCVLSGHTFYDTFDKARYQFQGPCKEILMAADCYWNTWDVKVSHRDVESYTEVEKVTIRKQSTVVDLIVDGKQVKVGGVDVSIPYSSENTSIYWQDGDILTTAILPEALVVKFNFKQLLVVHIRDPFDGKTCGICGNYNQDSTDDFFDAEGACALTPNPPGCTEEQKPEAERLCNNLFDSSIDEKCNVCYKPDRIARCMYEYCLRGQQGFCDHAWEFKKECYIKHGDTLEVPPECQGSTEPGLEEVGEIEQKQLQKRFGGFTGARKSARKLANQGSVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 3 - protein, 16 aa, mammalian
SEQKQLQKRFGGFTGG

SEQ ID NO: 4 - protein, 16 aa, mammalian
SEQKQLQGGFGGFTGG

SEQ ID NO: 5 - protein, 14 aa, mammalian
SEQKQLQFGGFTGG